(19) United States
(12) Patent Application Publication
(10) Pub. No.: US 2005/0129571 A1
(43) Pub. Date: Jun. 16, 2005

(54) Ozone enhanced vaporized hydrogen peroxide decontamination method and system

(75) Inventor: Michael A. Centanni, Parma, OH (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 10/734,059

(22) Filed: Dec. 10, 2003

(51) Int. Cl. A61L 2/20

(52) U.S. Cl. 422/31; 422/28; 422/30; 422/292; 422/305; 422/62; 422/3

(57) ABSTRACT

A vapor decontamination system for decontaminating a defined region. The system is comprised of a chamber defining a region, a generator for generating vaporized hydrogen peroxide from a solution of hydrogen peroxide and water and a device for the introduction of ozone. A closed loop circulating system is provided for supplying the vaporized hydrogen peroxide and the ozone to the region. A destroyer breaks down the vaporized hydrogen peroxide. A sensor and a controller controlling the device for the introduction of ozone are provided to maintain the ozone at the desired concentration.

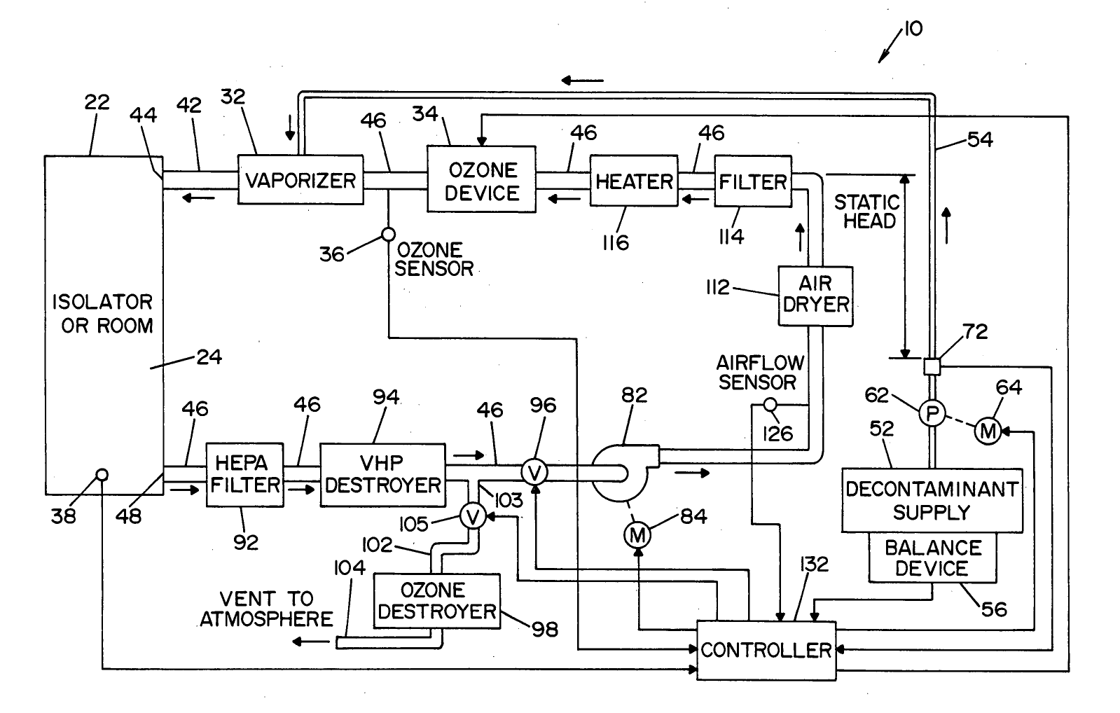

### FIELD OF THE INVENTION

The present invention relates generally to the art of sterilization and decontamination, and more particularly to a decontamination method and system that includes the sequential or simultaneous application of ozone and vaporized hydrogen peroxide

### BACKGROUND OF THE INVENTION

Decontamination methods are used in a broad range of applications, and have used an equally broad range of sterilization agents and decontamination agents. As used herein, the term “sterilization” refers to the inactivation of all bio-contamination, especially on inanimate objects. “Decontamination” refers to the inactivation of all vegetative biological agents, especially on inanimate objects. The term “disinfectant” refers to the inactivation of organisms considered pathogenic. The term “decontaminant” refers to a decontaminating agent.

The use of vaporized hydrogen peroxide (VHP) for sterilization is known. Known methods of sterilization with VHP include open loop systems and closed loop systems. In a known closed loop system, a carrier gas, such as air, is dried and heated prior to flowing past a vaporizer. A hydrogen peroxide aqueous solution is introduced into the vaporizer and vaporized. The resulting vapor is then combined with the carrier gas and introduced into a sterilization chamber. A blower exhausts the carrier gas from the sterilization chamber and recirculates the carrier gas to the vaporizer where additional VHP is added. Between the sterilization chamber and the vaporizer, the recirculating carrier gas passes through a catalytic destroyer (where any remaining VHP is eliminated from the carrier gas), a drier, a filter and a heater.

It is also known to sterilize and decontaminate with ozone using an ozone sterilizer. Ozone sterilizers utilize an ozone generator or other device to introduce ozone into a carrier gas. A typical carrier gas for ozone sterilization is atmospheric air. After the addition of ozone to the carrier gas, the carrier gas is introduced into the sterilization chamber or room to be sterilized. The ozone acts by oxidizing any bio-contamination exposed to the ozone thereby inactivating the bio-contamination. The ozone also acts as a bleaching agent. Ozone in a humid environment has greater bleaching properties than other known bleaching agents such as hydrogen peroxide, chlorine, or sulfur dioxide.

The present invention provides a method and system for decontamination using a combination of VHP and ozone.

### SUMMARY OF THE INVENTION

In accordance with a preferred embodiment of the present invention, there is provided a vapor decontamination system for decontaminating a defined region. The system is comprised of a chamber defining a region, a generator for generating vaporized hydrogen peroxide from a solution of hydrogen peroxide and water, and a device for introducing ozone into a carrier gas. A closed loop circulating system is provided for supplying a vaporized hydrogen peroxide and ozone mixture to the region. A destroyer breaks down the vaporized hydrogen peroxide discharged from the region.

In accordance with another aspect of the present invention, there is provided a decontamination system for decontaminating a region. The decontamination system has a generator for generating vaporized hydrogen peroxide, and a generator for generating ozone. A closed loop system is provided for supplying the vaporized hydrogen peroxide and ozone to the region. A destroyer is provided for breaking down the vaporized hydrogen peroxide into water and oxygen.

In accordance with yet another aspect of the present invention, there is provided a closed loop, flow-through method of vapor phase decontamination in a sealable chamber or region having an inlet port and an outlet port, and a closed loop conduit fluidly connecting the outlet port to the inlet port. The method comprises the steps of:
generating a flow of a carrier gas into, through and out of the chamber, and through the closed loop conduit;
supplying ozone to the carrier gas flow;
supplying vaporized hydrogen peroxide to the carrier gas flow; and
destroying the vaporized hydrogen peroxide at a first location downstream from the outlet port to form water and oxygen.

In accordance with yet another aspect of the present invention, there is provided a closed loop, flow through vapor phase decontamination system, comprised of a sealable chamber having an inlet port and an outlet port. A closed loop conduit system has a first end fluidly connected to the inlet port and a second end fluidly connected to the outlet port. A blower is connected to the conduit system for re-circulating a carrier gas flow into, through and out of the chamber. A vaporizer is provided for delivering vaporized hydrogen peroxide into the carrier gas flow upstream of the inlet port. An ozone generator is provided upstream of the vaporizer. A destroyer downstream of the outlet port converts the vaporized hydrogen peroxide into water and oxygen.

An advantage of the present invention is a decontamination system that combines the decontamination aspects of vaporized hydrogen peroxide and ozone.

Another advantage of the present invention is a system as defined above that can utilize only vaporized hydrogen peroxide.

Another advantage of the present invention is a system as defined above that can utilize only ozone.

A further advantage of the present invention is a system as defined above that effectively combines ozone and vaporized hydrogen peroxide where ozone alone would cause degradation to the device being sterilized.

A further advantage of the present invention is the provision of a system as defined above that effectively minimizes the costs associated with decontamination with vaporized hydrogen peroxide alone by combining vaporized hydrogen peroxide with ozone.

A still further advantage of the present invention is a system as defined above that is operable to decontaminate with either: 1) vaporized hydrogen peroxide 2) ozone or 3) a combination of vaporized hydrogen peroxide and ozone.

A still further advantage of the present invention is a system as defined above where ozone is generated in dry conditions thereby promoting the production of ozone.

A still further advantage of the present invention is a system as defined above where ozone is exposed to objects to be decontaminated in humid conditions thereby promoting the bleaching qualities of ozone.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

The FIGURE is a schematic view of an ozone enhanced vaporized hydrogen peroxide decontamination system.

### DETAILED DESCRIPTION OF PREFERRED EMBODIMENT

Referring now to the drawings wherein the showings are for the purpose of illustrating a preferred embodiment of the invention only, and not for the purpose of limiting same, the FIGURE shows a decontamination system 10, illustrating a preferred embodiment of the present invention. Broadly stated, system 10 utilizes a combination of vaporized hydrogen peroxide (i.e., a two-component, vapor-phase decontaminant) and ozone for decontaminating a space or region, or articles within the space or region.

In the embodiment shown, system 10 includes an isolator or room 22 that defines an inner sterilization/decontamination chamber or region 24. It is contemplated that articles to be sterilized or decontaminated may be disposed within isolator or room 22. A supply conduit 42 defines a decontaminant inlet 44 to chamber or region 24. Supply conduit 42 connects a vaporizer 32 to sterilization/decontamination chamber or region 24 of isolator or room 22. Vaporizer 32 is connected to a liquid decontaminant supply 52 by a feed line 54. A conventionally known balance device 56 is associated with decontaminant supply 52, to measure the actual mass of decontaminant being supplied to vaporizer 32.

A pump 62, driven by a motor 64, is provided to convey metered amounts of the liquid decontaminant to vaporizer 32 where the decontaminant is vaporized by conventionally known means. In an alternate embodiment, pump 62 is provided with an encoder (not shown) that allows monitoring of the amount of decontaminant being metered to vaporizer 32. If an encoder is provided with pump 62, balance device 56 is not required. A pressure switch 72 is provided in feed line 54. Pressure switch 72 is operable to provide an electrical signal in the event that a certain static head pressure does not exist in feed line 54. A VHP sensor 38 is disposed within sterilization/decontamination chamber or region 24 of isolator or room 22 for determining the concentration of VHP therein.

Isolator or room 22 and vaporizer 32 are part of a closed loop system that includes a return conduit 46 that connects isolator or room 22 (and sterilization/decontamination chamber or region 24) to vaporizer 32. Return conduit 46 defines a decontaminant outlet 48 to sterilization/decontamination chamber or region 24. A blower 82, driven by a motor 84, is disposed within return conduit 46 between isolator or room 22 and vaporizer 32. Blower 82 is operable to circulate decontaminant and a carrier gas such as air through the closed loop system.

A first filter 92, a VHP destroyer 94, and a valve 96 are disposed in return conduit 46 between blower 82 and isolator or room 22, as illustrated in the FIGURE. First filter 92 is preferably a HEPA filter and is provided to remove contaminants flowing through system 10. VHP destroyer 94 is operable to destroy hydrogen peroxide (H₂O₂) flowing therethrough, as is conventionally known. VHP destroyer 94 converts the hydrogen peroxide (H₂O₂) into water and oxygen. Valve 96 is operable to control flow through return conduit 46. Valve 96 is movable between a first position allowing flow through return conduit 46 and a second position blocking or preventing flow through return conduit 46.

In a preferred embodiment, an ozone destroyer 98 is disposed in a supplemental conduit 102. Ozone destroyer 98 is operable to destroy ozone (O₃), as is conventionally known. Ozone destroyer 98 may be any device that reduces the concentration of ozone relative to the carrier gas. In a preferred embodiment, ozone destroyer 98 is comprised of activated carbon. Ozone molecules that come in contact with the carbon surface react to form carbon dioxide (carbon monoxide is a secondary product) via direct chemical oxidation.

Supplemental conduit 102 has a first end 103 fluidly connected to return conduit 46 and a second end 104 open to the atmosphere. First end 103 of supplemental conduit 102 is fluidly connected to return conduit 46 between valve 96 and VHP destroyer 94. In a preferred embodiment, a valve 105 is disposed in supplemental conduit 102 between first end 103 and ozone destroyer 98. Valve 105 is operable to control flow through return supplemental conduit 102. Valve 105 is movable between a first position allowing flow through supplemental conduit 102 and a second position blocking or preventing flow through supplemental conduit 102. Second end 104 of supplemental conduit 102 is open and allows the contents of supplemental conduit 102 to vent to the atmosphere. It is recognized that second end 104 may be fluidly connected to return conduit 46 between valve 96 and blower 82.

An air dryer 112, filter 114 and heater 116 are disposed within return conduit 46 between blower 82 and vaporizer 32. Air dryer 112 is operable to remove moisture from air blown through the closed loop system. Second filter 114 is operable to filter the air blown through return conduit 46 by blower 82. Heater 116 is operable to heat air blown through return conduit 46 by blower 82. In this respect, air is heated prior to the air entering vaporizer 32.

An airflow sensor 126 is disposed in return conduit 46 between blower 82 and air dryer 112. Airflow sensor 126 is operable to sense the airflow through return conduit 46.

In accordance with one aspect of the present invention, an ozone device 34 is disposed in return conduit 46 between heater 116 and vaporizer 32. Ozone device 34 is provided to introduce gaseous ozone into return conduit 46. In a preferred embodiment, ozone device 34 is a generator for generating ozone. Known devices for generating ozone utilize various types of energy sources such as electrochemical, electromagnetic (e.g., ultraviolet light, laser light, and electron beam), and electrical. In a preferred embodiment, ozone device 34 is an electrical device, namely a corona discharge device such as the device described in U.S. Pat. No. 3,872,313 to Emigh et al. It is also recognized that ozone device 34 could be a device for the input of gaseous ozone from an external source. Such an external source could be a storage container where ozone is stored (for example, in a compressed, liquefied state) or an external ozone generator. Ozone device 34 is dimensioned to generate, supply, or introduce ozone at a rate sufficient to maintain the concentration of ozone within sterilization/decontamination chamber or region between 1 ppm and 500 ppm. Preferably, ozone device 34 is dimensioned to generate, supply, or introduce ozone at a rate sufficient to maintain the concentration of ozone within sterilization/decontamination chamber or region between 1 ppm and 100 ppm. More preferably, ozone device 34 is dimensioned to generate, supply, or introduce ozone at a rate sufficient to maintain the concentration of ozone within sterilization/decontamination chamber or region between 1 ppm and 50 ppm. Ozone device 34 is connected by a control signal to a controller 132.

An ozone sensor 36 is disposed in return conduit 46 between ozone device 34 and vaporizer 32. Ozone sensor 36 is operable to sense the concentration of ozone within return conduit 46. In a preferred embodiment, ozone sensor 36 may be one of several known devices for sensing ozone. Ozone sensor 36 is electrically connected to a controller 132. It is contemplated that ozone sensor 36 could be disposed at any location in return conduit 46, supply conduit 42, or sterilization/decontamination chamber or region 24. It is further contemplated that a plurality of ozone sensors could be disposed in return conduit 46, supply conduit 42, or sterilization/decontamination chamber or region 24.

In the embodiment shown, ozone sensor 36, VHP sensor 38, and airflow sensor 126 provide electrical signals to a system controller 132 that is schematically illustrated in the FIGURE. Controller 132 is a system microprocessor or micro-controller programmed to control the operation of system 10. Controller 132 is programmed to monitor and control the desired concentrations of VHP and ozone based upon programmed control parameters. The control parameters used may be expressed as a desired VHP concentration and a desired ozone concentration or as a ratio of VHP to ozone. As illustrated in the FIGURE, controller 132 is also connected to motor 64, motor 84, pressure switch 72, balance device 56, ozone device 34, valve 96 and valve 105.

The present invention shall now be further described with reference to the operation of system 10. A typical sterilization/decontamination cycle includes a drying phase, a conditioning phase, a decontamination phase and an aeration phase. Prior to running a sterilization/decontamination cycle, data regarding the percent of hydrogen peroxide in the decontaminant solution and the desired concentration of ozone at sensor 36 is entered, i.e., inputted, into controller 132. As noted above, in a preferred embodiment, a decontaminant solution of 35% hydrogen peroxide and 65% water is used. However, other concentrations of hydrogen peroxide and water are contemplated.

Isolator or room 22, supply conduit 42 and return conduit 46 define a closed loop conduit circuit. When a sterilization/decontamination cycle is first initiated, controller 132 causes blower motor 84 to drive blower 82, thereby causing a carrier gas to circulate through the closed loop circuit. In a preferred embodiment, the carrier gas is air. During a drying phase, vaporizer 32 and ozone device 34 are not operating. Air dryer 112 removes moisture from the carrier gas (i.e., air) circulating through the closed loop system, that is, through supply conduit 42, return conduit 46 and sterilization/decontamination chamber or region 24 or isolator or room 22, as illustrated by the arrows in the FIGURE. When the air has been dried to a sufficiently low humidity level, the drying phase is complete. It is contemplated that the desired humidity levels will be chosen according to the combination of ozone and VHP to be used and the effect desired.

The conditioning phase is then initiated by activating vaporizer 32 and decontaminant supply motor 64 to provide decontaminant to vaporizer 32. In a preferred embodiment, the decontaminant supplied to vaporizer 32 is a hydrogen peroxide solution comprised of about 35% hydrogen peroxide and about 65% water. A decontaminant solution comprised of other ratios of hydrogen peroxide and water is also contemplated. Within vaporizer 32, the liquid decontaminant is vaporized to produce vaporized hydrogen peroxide (VHP) and water vapor in a conventionally known manner. The vaporized decontaminant is introduced into the closed loop conduit circuit and is conveyed through supply conduit 42 by the carrier gas (air) into sterilization/decontamination chamber or region 24 within isolator or room 22.

During the conditioning phase, VHP is conveyed by the carrier gas into sterilization/decontamination chamber or region 24 to bring the VHP level up to a desired level in a short period of time. During the conditioning phase, blower 82 causes air to continuously circulate through the closed loop system. As the carrier gas enters chamber or region 24 from vaporizer 32, the carrier gas is also being drawn out of chamber or region 24 through VHP destroyer 94 where VHP is broken down into water and oxygen.

After the conditioning phase is completed, the decontamination phase is initiated. Ozone generation is initiated and maintained at a desired level by system controller 132. System controller 132 controls the introduction of ozone by controlling the output of ozone device 34.

Ozone device 34 generates ozone by the corona discharge method. The corona discharge method produces ozone by subjecting a gas that contains oxygen molecules (i.e. the carrier gas) to electrical charges. The carrier gas is passed through a discharge gap defined by a first electrode and a second electrode. A voltage differential is developed between the two electrodes thereby causing electrons to pass through a dielectric on the first electrode and cross the discharge gap from the first electrode to the second electrode. The flow of electrons from the first electrode to the second electrode is a corona discharge. A corona discharge is characterized by a low current electrical discharge at a voltage gradient that exceeds a certain critical value. The corona discharge provides the energy to disassociate the oxygen molecules contained within the carrier gas. The resulting oxygen atoms combine with the remaining oxygen molecules to form ozone. Dry conditions promote the production of ozone by preventing “leaking” of electrons that reduces the desired voltage gradient and can be caused by humid conditions. By placing ozone device 34 downstream of air dryer 112, humidity resulting from the breakdown of VHP or from other sources is removed prior to the production of ozone thus providing non-humid dry conditions that promote the production of ozone.

Controller 132 monitors the signal returned by ozone sensor 36, compares that signal with the programmed control parameters, i.e., the desired concentration of ozone, and adjusts the amount of ozone introduced by ozone device 34 into the carrier gas accordingly. Thus, ozone sensor 36, controller 132, and ozone device 34 operate as a closed-loop feedback ozone control system maintaining a desired concentration of ozone in the carrier gas in supply conduit 42. More specifically, ozone will degrade over time as it is transferred through supply conduit 42, return conduit 46 and sterilization/decontamination chamber or region 24 or isolator or room 22, as illustrated by the arrows in the FIGURE. Any ozone that is not consumed or degraded during the decontamination process while it is transferred through system 10 is supplemented with ozone introduced into return conduit 46 by ozone device 34.

The decontamination phase is continued for a predetermined period of time sufficient to effect the desired sterilization or decontamination of sterilization/decontamination chamber or region 24, and items therein. It is preferred to maintain the hydrogen peroxide and ozone concentrations within desired limits that may be defined by one skilled in the art as necessary to achieve the desired degree of decontamination. Blower 82 circulates VHP and ozone as described above.

Also during the decontamination phase, the atmosphere within sterilization/decontamination chamber or region 24 of isolator or room 22 contains water vapor produced by the vaporization of the liquid contaminant within vaporizer 32 and by the degradation of VHP. The resulting humidity within the atmosphere of sterilization/decontamination chamber or region 24 promotes the bleaching properties of the ozone within sterilization/decontamination chamber or region 24.

It is believed that the actual amount of hydrogen peroxide used during a given decontamination/sterilization cycle in combination with ozone will be less than the amount of hydrogen peroxide used if only hydrogen peroxide were used during an otherwise identical decontamination/sterilization cycle.

After the decontamination phase is completed, controller 132 causes vaporizer 32 and ozone device 34 to shut down, thereby shutting off both the introduction of decontaminant to supply conduit 42 and the introduction of ozone to return conduit 46.

Thereafter, the aeration phase is initiated to bring the hydrogen peroxide level down to an allowable threshold (about 1 ppm). In this respect, blower 82 continues to circulate the air, remaining VHP, and remaining ozone through the closed loop system. Eventually all of the vaporized hydrogen peroxide (VHP) will be delivered to VHP destroyer 94 and will be broken down. Since ozone is an unstable molecule at normal atmospheric conditions, it will naturally break down over time. The aeration phase preferably lasts for a sufficient period to allow for satisfactory breakdown of the ozone within system 10.

In another preferred embodiment, it is contemplated that valve 96 and valve 105 are operated so that flow through return line 46 be directed through ozone destroyer 98 after passing through VHP destroyer 94 and before being vented to the atmosphere. It is recognized that various other conduit and valve arrangements can be utilized to direct the contents of return line 46 to flow through ozone destroyer 98. In addition, it is recognized that the flow through return line 46 can be directed back into return line 46 after having passed through ozone destroyer 98 (not shown).

The foregoing description is a specific embodiment of the present invention. It should be appreciated that this embodiment is described for purposes of illustration only, and that those skilled in the art may practice numerous alterations and modifications without departing from the spirit and scope of the invention.

Among those modifications, an alternate method of using system 10 as described above is contemplated whereby only VHP is used as a decontaminant. In this alternate embodiment, ozone device 34 is not operated and does not introduce ozone into return conduit 46. Ozone device 34 remains disposed within return conduit 46 between heater 116 and vaporizer 32 and the carrier gas propelled by blower 82 continues to be transferred through ozone device 34. Controller 132 is programmed so that ozone device 34 does not operate and does not introduce ozone into return conduit 46.

A further alternate method of using system 10 as described above is contemplated whereby only ozone is used as a decontaminant. Ozone device 34 introduces ozone into return conduit 46 but vaporizer 32 does not introduce VHP into supply conduit 42. Vaporizer 32 remains connected to sterilization/decontamination chamber or room 24 of isolator or room 22 and the carrier gas propelled by blower 82 continues to transfer through vaporizer 32. However, controller 132 is programmed so that pump 62 driven by motor 64 does not convey the liquid decontaminant to vaporizer 32. It is recognized that motor 64 may be disabled in some additional manner including removal of electrical supply and that the flow of liquid decontaminant may be physically interrupted.

Another alternate method of using system 10 as described above is contemplated whereby oxygen or an oxygen-rich gas is introduced between heater 116 and ozone device 34 from a source (not shown) in order to enhance the amount of ozone produced in ozone device 34.

It is intended that all such modifications and alterations be included insofar as they come within the scope of the invention as claimed or the equivalents thereof.

1. A vapor decontamination system for decontaminating a defined region, said system comprising:
a chamber defining a region;
a first generator for generating vaporized hydrogen peroxide from a solution of hydrogen peroxide and water and introducing vaporized hydrogen peroxide into a carrier gas;
a device for introducing ozone into said carrier gas;
a closed loop circulating system for supplying said vaporized hydrogen peroxide and said carrier gas to said region; and
a destroyer for breaking down said vaporized hydrogen peroxide.

2. A vapor decontamination system as defined in claim 1, wherein said carrier gas comprises oxygen and said second generator generates ozone from said oxygen.

3. A vapor decontamination system as defined in claim 1, wherein a sensor is operable to detect the concentration of ozone in said carrier gas.

4. A vapor decontamination system as defined in claim 1, wherein said first generator is a vaporizer.

5. A vapor decontamination system as defined in claim 1, further comprising:
a destroyer for breaking down said ozone.

6. A vapor decontamination system as defined in claim 1, further comprising:
a blower within said closed loop circulating system, said blower operable to circulate air through said closed loop circulating system;
a dryer disposed within said closed loop circulating system between said destroyer and said generator, said dryer operable to remove moisture from said circulating system; and
a heater within said closed loop circulating system upstream from said first generator for heating air flowing through said circulating system.

7. In a decontamination system for decontaminating a region, said system having a first generator for generating vaporized hydrogen peroxide, a second generator for generating ozone, a closed loop system for supplying the vaporized hydrogen peroxide and the ozone to said region and a destroyer for breaking down the vaporized hydrogen peroxide, a sensor for detecting the concentration of ozone in said system, and a controller for determining the presence of ozone in said region based upon data from said sensor.

8. A decontamination system as defined in claim 7, wherein said controller is operable to determine the concentration of ozone in said region.

9. A decontamination system as defined in claim 8, wherein said sensor is an ozone sensor.

10. A method of sterilizing with a combination of ozone and vaporized hydrogen peroxide (VHP) in a region, comprising the steps of:
providing a sealable region having an inlet port and an outlet port, and a closed loop conduit having a first end fluidly connected to the region inlet port and a second end fluidly connected to the region outlet port;
re-circulating a flow of a carrier gas into, through and out of said region and around the closed loop conduit;
delivering vaporized hydrogen peroxide into the re-circulating carrier gas flow upstream of the region inlet port;
delivering ozone into the re-circulating carrier gas flow upstream of the region inlet port;
destroying the vaporized hydrogen peroxide at a first location downstream from the region outlet port; and
determining a presence of ozone in said region based upon the readings derived from an ozone sensor.

11. A method as defined in claim 10, wherein said carrier gas is air.

12. A method as defined in claim 10, wherein said delivering ozone includes the electrical generation of ozone from said carrier gas.

13. A closed loop, flow through method of vapor phase decontamination in a sealable chamber or region having an inlet port and an outlet port, and a closed loop conduit fluidly connecting the outlet port to the inlet port, the method comprising the steps of:
re-circulating a flow of a carrier gas into, through and out of the chamber, and through the closed loop conduit;
supplying vaporized hydrogen peroxide into the re-circulating carrier gas flow;
supplying ozone into the re-circulating carrier gas flow;
destroying the vaporized hydrogen peroxide to form water and oxygen at a first location downstream from said outlet port; and
monitoring the concentration of ozone within the carrier gas.

14. A closed loop, flow through method as defined in claim 13, wherein said carrier gas is air.

15. A closed loop, flow through method as defined in claim 13, wherein said destroying step includes catalytically decomposing the hydrogen peroxide vapor into water and oxygen.

16. A closed loop, flow through vapor phase decontamination system, comprising:
a sealable chamber having an inlet port and an outlet port;
a closed loop conduit system having a first end fluidly connected to said inlet port and a second end fluidly connected to said outlet port;
a blower connected to said conduit system for re-circulating a carrier gas flow into, through and out of the chamber;
a vaporizer for delivering vaporized hydrogen peroxide into said carrier gas flow upstream of said inlet port;
a generator for delivering ozone into said carrier gas flow upstream of said inlet port;
a destroyer downstream of said outlet port for converting the vaporized hydrogen peroxide in water and oxygen; and
a sensor for detecting ozone.
